(19) United States
(12) Patent Application Publication
(10) Pub. No.: US 2026/0263531 A1
(43) Pub. Date: Sep. 10, 2026

(54) DEGRADATION SYSTEM ACCELERATOR FOR RECYCLING ENHANCEMENT

(71) Applicant: FORDAYS CO., LTD., Tokyo (JP)

(72) Inventors: Keita SUTOH, Tokyo (JP); Mica FUJITA, Tokyo (JP)

(21) Appl. No.: 18/876,752

(22) PCT Filed: Jan. 16, 2024
(86) PCT No.: PCT/JP2024/000989

§ 371 Date: Dec. 19, 2024

(30) Foreign Application Priority Data
Oct. 3, 2023 (JP) 2023-172240

(51) Int. Cl. A61K 35/60

(52) U.S. Cl. CPC A61K 35/60

(57) ABSTRACT

A degradation system accelerator for recycling enhancement contains a water-soluble nuclear protein, in particular, a water-soluble nuclear protein as a low-molecular-weight substance with a molecular weight of 250 to 15,000 obtained by treating fish milt with an enzyme. A health food containing the degradation system accelerator. A method of increasing responses of various degradation system-related genes and a recycling enhancement method for cells and the like conducted by orally ingesting the degradation system accelerator.

### TECHNICAL FIELD

The present invention relates to a degradation system related to recycling enhancement of cells and the like, and specifically, relates to a degradation system accelerator that accelerates a degradation system, such as proteasome, autophagy, and apoptosis, to enhance the recycling of cells, cell organelles, and the like.

The present invention also relates to a method of increasing responses of various degradation system-related genes (proteasome-related genes, autophagy-related genes, and apoptosis-related genes) by orally ingesting the degradation system accelerator.

### BACKGROUND ART

Reflecting the growing public interest in health in recent years, health foods using a deoxyribonucleic acid (DNA), a ribonucleic acid (RNA), and a nuclear protein (also referred to as a nucleoprotein and a salmon milt extract), which is a complex of these nucleic acids and proteins, as a raw material or active ingredient have been provided. For example, nuclear proteins have been known to have an immunoregulatory action, an antioxidant action, and a vasodilatory action. In addition, there has been a proposal of a cancer cell proliferation inhibiting agent containing a water-soluble nucleic acid with a lowered molecular weight or the water-soluble nucleic acid and an amino acid (Patent Document 1).
Patent Document 1: JP-A-2014-152144

### DISCLOSURE OF THE INVENTION

### Problems to be Solved by the Invention

Until now, it has been introduced that the ingestion of nucleic acids (DNA, RNA) and nuclear proteins has resulted in an onset of various actions and effects, including antioxidant action.

However, it has not yet been reported that the ingestion affects the degradation of aging cells, unwanted oxidized proteins in cells, and defective mitochondria, thereby affecting the recycling of the cells themselves, cell organelles, and the like.

As degradation systems for recycling in cells, a ubiquitin-proteasome system and autophagy exist. The ubiquitin-proteasome system selectively degrades unwanted proteins and damaged proteins in cells. Autophagy is associated with vesicular autophagosomes involved in self-degradation of similarly damaged or dysfunctional subcellular organelles. In addition, selectively removing particularly damaged mitochondria is referred to as mitophagy. While a target to be degraded in autophagy is “non-selective,” the above-described ubiquitin-proteasome system selectively degrades by marking a protein that is mainly degraded with a ubiquitin protein. A recent study has reported that these phenomena are related to aging and diseases. For example, it has been reported that in Drosophila, a decrease in average life expectancy is observed when a mutation enters an autophagy gene, while overexpression leads to an increase in average life expectancy. Moreover, the relationship between autophagy and neurodegenerative diseases, such as Alzheimer's dementia and Parkinson's disease, has been recognized, and autophagy failure has been reported to cause accumulation of amyloid proteins, such as β-amyloid and α-synuclein.

However, it has not yet been reported that the ubiquitin-proteasome system and autophagy are associated with antioxidation.

An object of the present invention is to provide a new formulation related to recycling enhancement of cells and the like and a method of increasing responses of various related genes related to the recycling enhancement of cells and the like.

### Solutions to the Problems

The present invention relates to a degradation system accelerator for recycling enhancement, the degradation system accelerator contains a water-soluble nuclear protein, and in particular relates to the degradation system accelerator in which the water-soluble nuclear protein is a low-molecular-weight nuclear protein with a molecular weight of 250 to 15,000 obtained by treating fish milt with an enzyme. Especially, the present invention relates to the degradation system accelerator in which to be intended for a proteasome degradation system, an autophagy degradation system, or apoptosis.

The present invention relates to a health food containing the degradation system accelerator and relates to the health food having a form of a tablet, drinkable preparation, capsule preparation, granule preparation, powdered preparation, pill, or jelly preparation.

The present invention relates to a method of increasing a response of a proteasome-related gene, a method of increasing a response of an autophagy-related gene, or a method of increasing a response of an apoptosis-related gene by orally ingesting the degradation system accelerator, and also relates to a recycling enhancement method for a cell, a cell organelle, or a protein by orally ingesting the degradation system accelerator.

The present invention relates to a response increasing agent for a proteasome-related gene, a response increasing agent for an autophagy-related gene, or a response increasing agent for an apoptosis-related gene containing a water-soluble nuclear protein, and further relates to a recycling enhancer for a cell, a cell organelle, or a protein, the recycling enhancer containing a water-soluble nuclear protein.

### Effects of the Invention

The degradation system accelerator of the present invention containing a water-soluble nuclear protein, in particular, a nuclear protein with a lowered molecular weight by treating fish milt with an enzyme, provides an effect of accelerating proteasome activation, autophagy activation, and apoptosis activation by orally ingesting the degradation system accelerator and accelerating the degradation and removal of oxidized proteins and cell organelles, such as defective mitochondria, and further, cell death itself to obtain a recycling enhancement action of these cells and the like. Furthermore, it is expected to contribute to health maintenance, such as antioxidation and disease prevention, through the progress of the recycling enhancement action.

### BRIEF DESCRIPTION OF THE DRAWINGS

FIG. 1 is a drawing showing the results of a d-ROMs test conducted with blood of test subjects before and after ingestion of tablets in a test tablet group and a placebo tablet group (A) trend in d-ROMs (measured value), (B) amount of change in d-ROMs (amount of change based on pre-ingestion)). In the test tablet group, test tablets containing a water-soluble nuclear protein (salmon milt extract) were ingested by test subjects for eight weeks. In the placebo tablet group, placebo tablets that did not contain the extract were ingested by test subjects for eight weeks.

FIG. 2 is a drawing showing the results of comprehensive gene expression analysis with a microarray using Total RNA extracted from human blood in human test subjects after eight weeks of ingestion of the water-soluble nuclear protein (salmon milt extract).

FIG. 3 is a drawing showing yeast in a test group and a control group that survived under hydrogen peroxide after 72 hours of culture at 30° C. In the test group, yeast (Saccharomyces cerevisiae BY4741 strain (MATa; his3D1; leu2D0; met15D0; ura3D0)) was enriched in an SD complete liquid culture medium to obtain a culture solution, and the water-soluble nuclear protein (salmon milt extract) was added to the culture solution so as to be 1% in amount. In the control group, yeast (Saccharomyces cerevisiae (BY4741 strain)) alone was enriched in an SD complete liquid culture medium to obtain a culture solution. The 72 hours of culture was conducted in the test group and the control group after appropriately diluting the culture solution after 24 hours of treatment and seeding it into a YPD flat plate culture medium to which 4 mM hydrogen peroxide was added.

FIG. 4 is a drawing showing survival rates for yeast in a test group and a control group obtained by counting the number of surviving yeast (number of colonies) with hydrogen peroxide added or not added after 72 hours of culture at 30° C. In the test group, yeast (Saccharomyces cerevisiae (BY4741 strain)) was enriched in an SD complete liquid culture medium to obtain a culture solution, and the water-soluble nuclear protein (salmon milt extract) was added to the culture solution so as to be 1% in amount. In the control group, yeast (Saccharomyces cerevisiae (BY4741 strain)) alone was enriched in an SD complete liquid culture medium to obtain a culture solution. The 72 hours of culture was conducted in the test group and the control group after appropriately diluting the culture solution after 24 hours of treatment and seeding it into each of a YPD flat plate culture medium to which 4 mM hydrogen peroxide was added and a YPD flat plate culture medium to which hydrogen peroxide was not added.

FIG. 5 is a drawing showing the results of comprehensive gene expression analysis with a microarray of Total RNA extracted from yeast (Saccharomyces cerevisiae ((BY4741 strain)) after a lapse of 24 hours after adding the water-soluble nuclear protein (salmon milt extract) to the yeast.

### DESCRIPTION OF PREFERRED EMBODIMENTS

A degradation system accelerator of the present invention contains a water-soluble nuclear protein. The inventors newly confirmed that the water-soluble nuclear protein activates proteasome, autophagy, and apoptosis, which are degradation systems of intracellular components and a cell itself, that is, the water-soluble nuclear protein has a function of degrading and removing defective proteins and defective cell organelles, such as oxidized proteins, carbonylated proteins, defective mitochondria, and impaired lysosomes, and further accelerating cell death itself. Therefore, the degradation system accelerator of the present invention can be expected to remove the above-described defective proteins, defective cells, cell organelles, and the like by orally ingesting the degradation system accelerator into a body to contribute to recycling these cells and the like.

The following describes the present invention in detail.

In the degradation system accelerator of the present invention, intended degradation systems are a proteasome degradation system, an autophagy degradation system, and apoptosis. These can be said, in other words, to be differences in the scale of degradation systems. The proteasome is associated with selective degradation of defective proteins, the autophagy is associated with degradation of defective proteins and defective subcellular organelles in cytoplasm, and the apoptosis is associated with degradation (cell death) of a cell itself. Accelerating the degradation of these defective cells and the like and configuring them to be easily recycled into new cells and the like can lead to recycling enhancement of cells and the like. Furthermore, as described above, the proteasome system and autophagy are involved in aging and various diseases, and a mutation and a decrease in these degradation systems lead to undesirable phenomena, such as a decrease in life expectancy. Therefore, the acceleration of the autophagy and proteasome system by ingesting nucleic acids (DNA, RNA) and nuclear proteins is considered to be able to expect not only an improvement of an antioxidation effect by simply ingesting the nucleic acids but also anti-aging and health maintenance.

Examples of the water-soluble nuclear protein used for the degradation system accelerator of the present invention, and a response increasing agent for proteasome-related genes, a response increasing agent for autophagy-related genes, and a response increasing agent for apoptosis-related genes, and a recycling enhancer for cells, cell organelles, or proteins, which are described later, can include a water-soluble nuclear protein obtained by extracting nucleic acids from a biological cell and lowering a molecular weight by enzyme treatment. That is, the water-soluble nuclear protein in this specification can be reworded as an extract of fish milt with a lowered molecular weight. Examples of the above-described water-soluble nuclear protein include a water-soluble nuclear protein (for example, a salmon milt extract) obtained by lowering the molecular weight of fish milt by enzyme treatment and the like to be water-soluble. In this specification, water-soluble means a property of being dissolvable in water at a concentration of 0.1 mass % or more.

Examples of the above-described fish include, for example, salmon, trout, and herring. Among these, salmon milt, which has not been used effectively as a resource in the past and has been discarded in large amounts despite containing many nucleic acids, can be preferably used.

Examples of the enzyme treatment of fish milt include, for example, a method in which a suspension liquid obtained by crushing the fish milt is treated with a protease and then treated with a nuclease. Normally, when DNA and the like are extracted from milt, a molecular weight is 1,000,000 or more. Therefore, the molecular weight is lowered by the enzyme treatment. Examples of a preferable water-soluble nuclear protein include those containing preferably 20 mass % or more, more preferably 30 mass % or more nucleoside/nucleotide/oligonucleotide/polynucleotide with a molecular weight of 250 to 100,000, for example, a molecular weight of 250 to 20,000, a molecular weight of 250 to 15,000, a molecular weight of 500 to 13,200, alternatively a molecular weight of 250 to 10,000, and a molecular weight of 250 to 6,600 (hereinafter also referred to as low-molecular-weight substances).

Further preferably, the water-soluble nuclear protein (for example, a salmon milt extract) according to the present invention can contain 2 mass % or more low-molecular-weight substances with a molecular weight of 500 to 15,000 and 18 mass % or more low-molecular-weight substances with a molecular weight of 250 to 6,600.

Examples of the protease can include, for example, serine protease, cysteine protease, metalloprotease, and aspartic protease. Since the fish milt contains proteins other than protamine, one having especially low substrate specificity is preferred. Examples of a good protease can include a protease manufactured by Novozymes Japan Ltd. Hydrolysis treatment using the protease can be conducted in a range of temperature: 30° C. to 60° C., for example, a range of 40° C. to 50° C., and in a range of pH: 6 to 7, considering the activation and deactivation of the protease.

The above-described nuclease hydrolyzes 3,5′-phosphodiester bond of deoxyribonucleic acid (DNA) to produce a 5′-nucleotide of oligomer polymerization. While the properties of the nuclease are not particularly limited, the nuclease preferably has a certain level of heat stability. Such a nuclease can be available, for example, as a commercial product from Amano Enzyme Inc., Sigma-Aldrich, and the like. Hydrolysis treatment using the nuclease can be conducted, for example, in a range of temperature: 60° C. to 75° C. and in a range of pH: 5 to 6, considering the activation and deactivation of the nuclease. As a specific procedure of the enzyme treatment, first, protease treatment is conducted on milt (for example, salmon milt), and proteins and the like contained in the milt are hydrolyzed. After the hydrolysis is completed, it is only necessary to lower the molecular weight of nucleic acids by conducting nuclease treatment.

The present invention is also intended for a health food containing the degradation system accelerator.

The health food of the present invention contains the degradation system accelerator as an active ingredient, for example, a water-soluble nuclear protein as a low-molecular-weight substance with about a molecular weight of 250 to 15,000, and for example, with about a molecular weight of 250 to 10,000 obtained by treating fish milt with an enzyme and contains other blended ingredients according to various product forms described below. An amount of the recycling agent contained in the health food is appropriately adjustable, referring to a clinical dosage described below.

The health food of the present invention can be made into a product that meets the preferences of users by mixing various ingredients, such as known sweeteners, acidulants, and vitamins. As the product form, the product can be provided, for example, in a formulation form of a tablet, capsule preparation, granule preparation, powdered preparation, pill, syrup preparation, liquid agent, emulsion, suspension liquid agent, or the like, and in a form of a drinkable preparation, jelly preparation, dairy product such as yogurt, seasoning, processed food, dessert, confectionery, or the like.

For example, the above-described tablet, capsule preparation, granule preparation, powdered preparation, pill, and the like for oral administration may be formulated by customary means, and these are prepared using an excipient, such as saccharose, lactose, glucose, starch, and mannitol; a binder, such as hydroxypropylcellulose, syrup, gum arabic, gelatin, sorbitol, tragacanth, methylcellulose, and polyvinylpyrrolidone; a disintegrant, such as starch, carboxymethylcellulose or calcium salt thereof, microcrystalline cellulose, and polyethylene glycol; a smoothing agent, such as talc, magnesium or calcium stearate, and silica; and a lubricant, such as sodium laurate and glycerol.

In addition, the above-described syrup preparation, liquid agent, emulsion, suspension liquid agent, and the like may also be formulated by customary means, and these are prepared using a solvent of the degradation system accelerator as an active ingredient, such as water, ethyl alcohol, isopropyl alcohol, propylene glycol, 1,3-butylene glycol, and polyethylene glycol; a surfactant, such as sorbitan fatty acid ester, polyoxyethylene sorbitan fatty acid ester, polyoxyethylene fatty acid ester, polyoxyethylene ether in hydrogenated castor oil, and lecithin; a suspension agent, for example, a cellulose derivative, such as carboxymethyl sodium salt and methylcellulose, and natural rubber, such as tragacanth and gum arabic; and a preservative, such as p-hydroxybenzoate ester, benzalkonium chloride, and sorbate.

Furthermore, the above-described drinkable preparation and jelly preparation, as well as the dairy product, seasoning, processed food, dessert, confectionery, and the like, may contain, in addition to raw materials constituting them, oligo-RNA, zinc, collagen, chondroitin, hyaluronan, a vitamin group (for example, a vitamin B family, such as vitamin B1, vitamin B2, vitamin B6, and vitamin B12, and vitamin C), glutathione (such as glutathione-containing yeast extract, reduced glutathione, and oxidized glutathione).

More specifically, for example, for use in drinks, but not limited to this use, pig collagen peptide, rare sugar-containing syrup, RNA-containing edible yeast extract, zinc-containing edible yeast, chondroitin-containing shark cartilage extract, hyaluronan-containing cockscomb extract/acidulant, vitamin C, arginine, magnesium carbonate, niacin, vitamin B6, vitamin B2, vitamin B1, folic acid, vitamin B12, anserine-containing fish meat extract, citrulline, salmon ovarian membrane extract, and pig placenta extract can also be blended. For example, for use in tablets, but not limited to this use, beer yeast, ginkgo leaf extract, garlic extract containing S-allylcysteine, and the like can also be blended. Furthermore, for example, for use in jelly preparations, but not limited to this use, fish collagen peptide and the like can also be blended.

In addition to the above-described degradation system accelerator as an active ingredient, the health food of the present invention may also contain other compounds and the like that are active in a medicinal or veterinary medicinal manner.

While the clinical dosage of the degradation system accelerator of the present invention or the like varies depending on age, weight, patient sensitivity, degree of symptom, and the like, a guideline for effective dosage can usually be approximately 100 mg to 500 mg per day for adults as an amount of, for example, the water-soluble nuclear protein (salmon milt extract). However, an amount outside of the above-described range can be used if necessary.

The above-described guideline for effective dosage also applies to the health food of the present invention. For the health food, an amount of the degradation system accelerator blended in the health food can be appropriately adjusted, for example, by referring to the above-described guideline for dosage per day and based on an amount of the health food itself ingested per day.

The degradation system accelerator of the present invention increases response genes associated with the degradation systems described above and accelerates these degradation systems.

That is, the present invention is also intended for a method of increasing responses of the proteasome-related genes, a method of increasing responses of the autophagy-related genes, and a method of increasing responses of the apoptosis-related genes by orally ingesting the degradation system accelerator. Since the degradation of cells and the like also enhances recycling, the present invention is also intended for a recycling enhancement method for cells, cell organelles, or proteins. The clinical dosage (guideline amount) and the like similar to those of the above-described degradation system accelerator can be directly and preferably applied to the methods of increasing the responses of these genes or the recycling enhancement method.

In addition, as described above, the present invention is intended for a response increasing agent for proteasome-related genes, a response increasing agent for autophagy-related genes, a response increasing agent for apoptosis-related genes, and a recycling enhancer for cells or cell organelles, which contain a water-soluble nuclear protein (for example, a salmon milt extract), and is further intended for a recycling enhancer for cells, cell organelles, or proteins.

These response increasing agents and recycling enhancers contain a water-soluble nuclear protein (for example, a salmon milt extract) as described above, and the water-soluble nuclear protein (for example, a salmon milt extract) used in the degradation system accelerator can be preferably used.

In addition, these response increasing agents and recycling enhancers can also be formed into health foods containing them. The health foods can preferably use the aspects, ingredients, and the like described for the health food containing the degradation system accelerator described above.

### EXAMPLES

### <Water-Soluble Nuclear Protein (Salmon Milt Extract)>

A water-soluble nuclear protein (salmon milt extract) obtained from salmon milt using hydrolytic enzymes, such as protease and nuclease, contained 20 mass % to 30 mass % nucleic acids (water-soluble nucleic acids in a molecular weight range of 250 to 15,000) and 50 mass % amino acids (including 15 mass % arginine).

### <Manufacturing of Test Tablets>

According to Table 1, test tablets containing the water-soluble nuclear protein (salmon milt extract) obtained in the above-described manufacturing example (Tablet A) and placebo tablets that did not contain the extract (Tablet B) were manufactured.

TABLE 1
[Table 1] Blend Prescription of Test Tablets

| | Tablet A | | | Tablet B | | |
|---|---|---|---|---|---|---|
| | Blending | Blending | | Blending | Blending | |
| | % | quantity* | | % | quantity * | |
| Water-soluble Nuclear | 24% | 0.36 | g | — | — | |
| Protein (Salmon Milt | | | | | | |
| Extract) | | | | | | |
| Dextrin | 62% | 0.93 | g | 86% | 1.29 | g |
| Crystalline Cellulose | 10% | 0.15 | g | 10% | 0.15 | g |
| Sucrose Ester | 2% | 0.03 | g | 2% | 0.03 | g |
| Calcium Stearate | 1% | 0.015 | g | 1% | 0.015 | g |
| Silicon Dioxide | 1% | 0.015 | g | 1% | 0.015 | g |
| Total | 100% | 1.5 | g | 100% | 1.5 | g |

*Blending quantity: Per 6 Tablets (1.5 g)

### Test Example (1): Change in Oxidative Stress Marker

A double-blind randomized placebo-controlled trial was conducted in 41 healthy subjects (men, not medicated, without underlying disease or the like) aged 40 or over but under 60 years.

The test subjects were divided into two groups, a test tablet group (Tablet A, 20 subjects) and a placebo tablet group (Tablet B, 21 subjects). In the respective test groups, Tablet A or Tablet B was ingested by the test subjects for eight weeks. In the test tablet group, Tablet A was prepared in quantity so that an amount of the water-soluble nuclear protein (salmon milt extract) ingested per day was 360 mg (6 tablets per day) and administered. The same quantity of Tablet B (6 tablets per day) as Tablet A was administered in the placebo tablet group.

Blood was collected from the test subjects before and after the ingestion (week 0 and week 8), and a Reactive Oxygen Metabolites-derived compounds (d-ROMs) test was conducted to comprehensively evaluate a state of oxidative stress level in vivo by measuring hydroperoxide as a metabolite caused by free radicals in blood.

In addition, Total RNA was extracted from the collected blood and used for gene expression analysis.

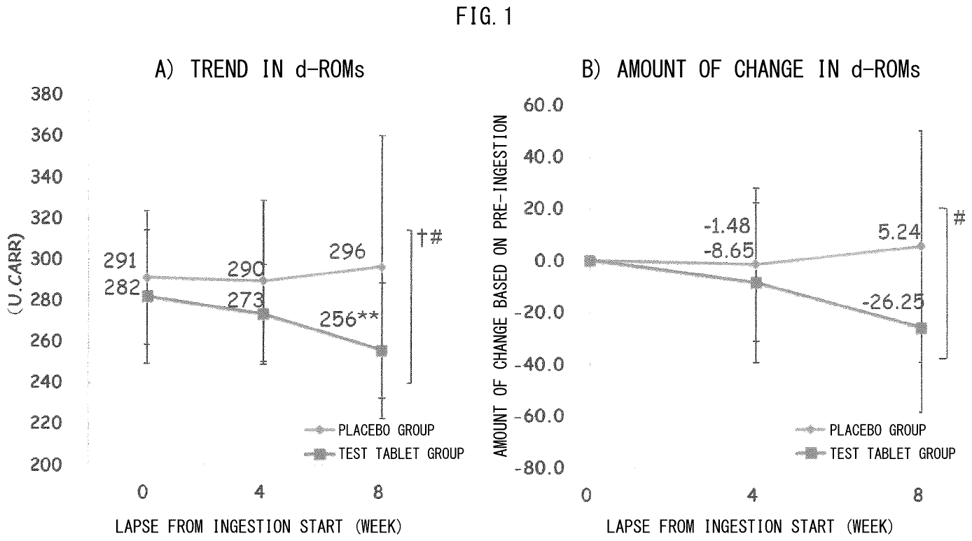

The results obtained are each shown in FIG. 1 [Results of d-ROMs test (FIG. 1(A) Measured value, FIG. 1(B) Amount of change based on pre-ingestion)].

As shown in FIG. 1, the test tablet group, in which Tablet A containing the water-soluble nuclear protein (salmon milt extract) was ingested for eight weeks, showed a significant decrease in the d-ROMs measured value and the amount of change (reduction of the oxidative stress marker) compared to the placebo tablet group.

### Test Example (2): Gene Expression Analysis

In a similar manner to Test Example (1), the respective tablets were ingested by the test subjects in the two groups of the test tablet group (Tablet A, 20 subjects) and the placebo tablet group (Tablet B, 21 subjects) for eight weeks. Then, Total RNA was extracted from the blood collected from the test subjects to conduct comprehensive gene expression analysis.

The comprehensive gene expression analysis conditions are as follows:

<Comprehensive Gene Expression Analysis with Microarray>

Microarray used: Applied Biosystems (registered trademark) Clariom S Array, Human manufactured by Thermo Fisher Scientific
1. An Agilent 2100 Bioanalyzer (Agilent Technologies) was used to conduct a quality check (RNA degradation evaluation) on Total RNA.
2. A sample with an RNA concentration of 100 ng/μl or more, an RNA amount of 300 ng or more, an A260/280 ratio of 1.8 to 2.1, and a RIN value of 7.0 or higher was used for testing.
3. A GeneChip WT Plus Reagent Kit was used to conduct cRNA synthesis. Using 100 ng of Total RNA as a template, first-strand cDNA in a first cycle was synthesized by reverse transcription reaction, and second-strand cDNA was further synthesized. Subsequently, cRNA was synthesized by in vitro transcription reaction. After purification, a yield of cRNA was measured by a spectrophotometer.
4. A GeneChip WT Plus Reagent Kit was used to synthesize single-strand cDNA from the cRNA. Single-strand cDNA in a second cycle was synthesized by reverse transcription reaction from the first cRNA. After purification, a yield of synthesized single-stranded DNA was measured by the spectrophotometer.
5. Fragmentation and labeling of the single-stranded DNA were conducted using a GeneChip WT Terminal Labeling Kit (Thermo Fisher Scientific). Using uracil DNA glycosylase (UDG) and apurinic/apyrimidinic endonuclear 1 (APE1), 5.5 μg of ssDNA was fragmented. Next, the fragmented ssDNA was biotin-labeled using terminal deoxynucleotidyl transferase (TdT) and a GeneChip DNA Labeling Reagent.
6. A GeneChip Hybridization, Wash, and Stain Kit (Thermo Fisher Scientific) was used to conduct hybridization to a Clariom S Array, Human. The fragmented and labeled ssDNA was added to a hybridization buffer, hybridized on the Clariom S Array, Human for 17 hours, washed, stained, and then scanned for fluorescence using a scanner to create a CEL file in GeneChip Command Console Software (GCC) and a CHP file in Expression Console™ Software.
7. Transcriptome Analysis Console (Thermo Fisher Scientific) was used to evaluate a change rate of a transcript at 1.3 or higher and conduct pathway analysis.

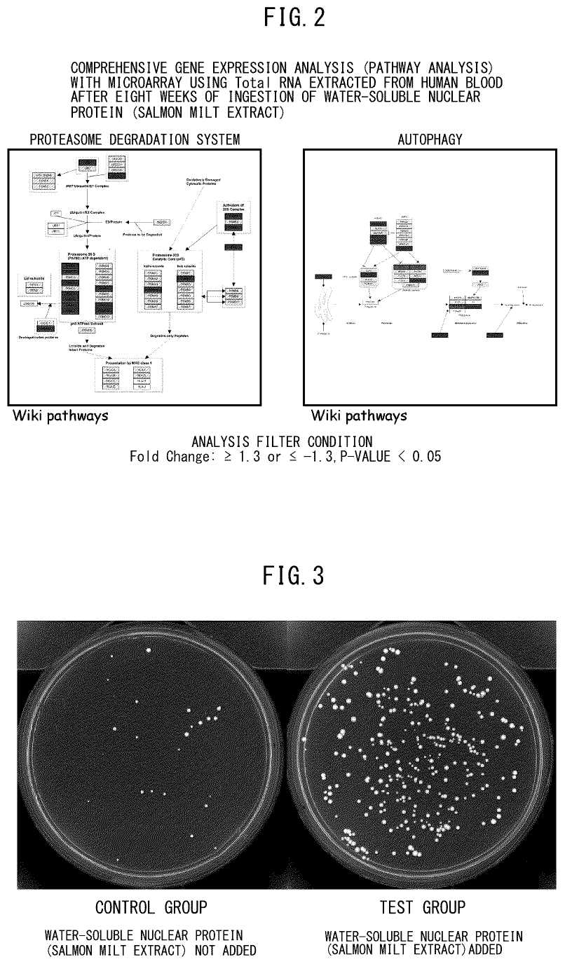

Table 2 shows the gene expression analysis results associated with various degradation systems (proteasome, autophagy, and apoptosis). FIG. 2 shows the results of the comprehensive gene expression analysis in the proteasome degradation system and autophagy.

TABLE 2
[Table 2] Gene Expression Analysis Results

| | | Gene Expression Level | |
|---|---|---|---|
| | | in Test Tablet Group | |
| | | [Ratio] | |
| Gene | | Versus | |
| Name | Protein Functionality | Placebo Group | P-value |

Proteasome-related Gene Expression

| | | | |
|---|---|---|---|
| UBE2D1 | Ubiquitin Complex Enzyme E2D1 | 2.07 | 0.0091 |
| PSMB3 | Proteasome Subunit β3 | 1.45 | 0.0472 |

Autophagy-related Gene Expression

| | | | |
|---|---|---|---|
| ATG7 | E1-like Activating Enzyme | 1.84 | 0.0122 |

Apoptosis-related Gene Expression

| | | | |
|---|---|---|---|
| DIABLO | IAP-binding Mitochondrial Protein | 1.39 | 0.0345 |
| CRADD | Death Domain-containing Protein | 1.40 | 0.0456 |
| IRF1 | Interferon Regulatory Factor 1 | 1.33 | 0.0353 |
| IRF2 | Interferon Regulatory Factor 2 | 1.32 | 0.0240 |

As shown in Table 2, it was confirmed that the test tablet group, in which Tablet A containing the water-soluble nuclear protein (salmon milt extract) was ingested for eight weeks, significantly increased the related gene expression of various degradation systems compared to the placebo tablet group.

### Test Example (3) Oxidative Stress Resistance Evaluation Using Yeast

Yeast (Saccharomyces cerevisiae) was used after isolating −80° C. stock of BY4741 strain on a flat plate, picking up two or three independent colonies, and conducting shake culture (160 rpm, 30° C.) in an SD complete culture medium for 18 hours to enrich the yeast.

A test group was prepared by adding a yeast culture solution in an amount of 1/10 to a new SD complete liquid culture medium and adding the above-described water-soluble nuclear protein (salmon milt extract) to the culture medium so as to be 1% of the culture medium. A control group was prepared only by adding the above-described yeast to an SD complete liquid culture medium (water-soluble nuclear protein (salmon milt extract) not added). Each was cultured for 24 hours. The yeast culture solution cultured for 24 hours was washed twice with sterile distilled water, and the water-soluble nuclear protein (salmon milt extract) was removed from the culture medium to make a sample. The yeast culture solution was appropriately diluted and then seeded into a YPD flat plate culture medium with hydrogen peroxide added so as to be 4 mM so that independent colonies could be obtained. After culture was conducted at 30° C. for 72 hours wards, the number of yeast colonies that survived in the presence of hydrogen peroxide was counted. FIG. 3 shows the appearances of the test group and the control group after the 72 hours of culture.

As shown in FIG. 3, more colonies were observed in the test group with the water-soluble nuclear protein (salmon milt extract) added than in the control group with the water-soluble nuclear protein (salmon milt extract) not added.

In addition, similarly to the above-described hydrogen peroxide-added system, for systems to which hydrogen peroxide was not added (with no hydrogen peroxide added) [a group of the yeast+the water-soluble nuclear protein (salmon milt extract), a group of the yeast only (with the water-soluble nuclear protein (salmon milt extract) not added)], culture was performed at 30° C. for 72 hours in a similar manner.

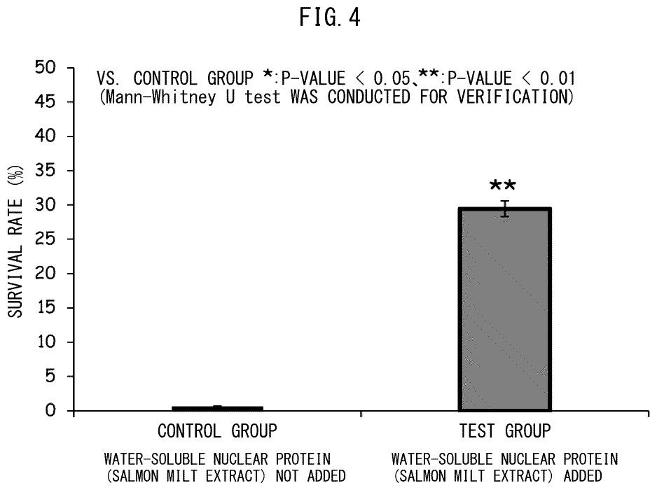

A yeast survival rate (%) was calculated by counting the number of growing colonies in each culture medium and dividing the number of colonies in the culture medium with hydrogen peroxide added by the number of colonies in the culture medium with no hydrogen peroxide added for each of the group with the water-soluble nuclear protein (salmon milt extract) added and the group with the water-soluble nuclear protein (salmon milt extract) not added. FIG. 4 shows the obtained results.

As shown in FIG. 4, the survival rate of the yeast to which the water-soluble nuclear protein (salmon milt extract) was added resulted in significantly higher than the survival rate of the yeast to which the water-soluble nuclear protein (salmon milt extract) was not added.

### Test Example (4) Comprehensive Gene Expression Analysis with Microarray Using Yeast

A comprehensive gene expression analysis was conducted with a microarray using Total RNA extracted from yeast (Saccharomyces cerevisiae (BY4741 strain (MATa; his3D1; leu2D0; met15D0; ura3D0)) after a lapse of 24 hours after adding the above-described water-soluble nuclear protein (salmon milt extract) in an amount of 1% to the yeast.

Microarray used: GeneChip Yeast Genome 2.0 Array from Thermo Fisher Scientific
1. An Agilent2200 TapeStaion was used to conduct a quality check (concentration measurement) of Total RNA.
2. Labeling reaction was conducted using a GeneChip (registered trademark) 3′ IVT PLIS Kit to conduct hybridization using 250 ng each of Total RNA.
3. An Expression Console™ was used to conduct quantification by RMA algorithm, and the quantified signal value was normalized by the quantile method.
4. Transcriptome Analysis Console (Thermo Fisher Scientific) was used to evaluate a change rate of a transcript at 1.3 or higher and conduct pathway analysis.

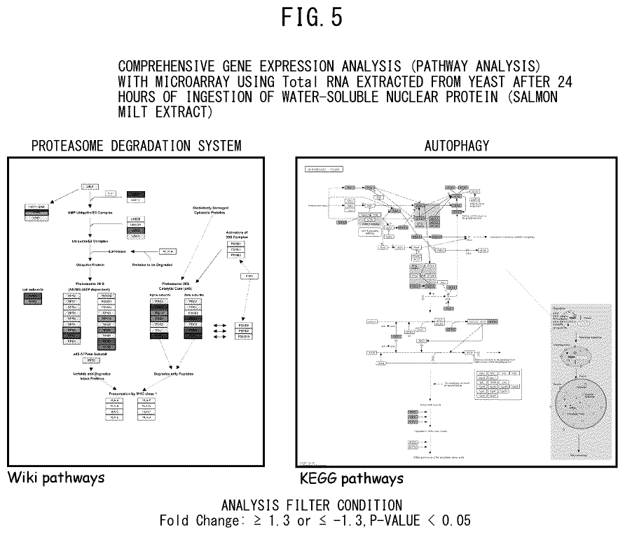

Table 3 and FIG. 5 show the gene expression analysis results associated with various degradation systems (proteasome and autophagy).

TABLE 3
[Table 3] Results of Gene Expression
Analysis Using Yeast (After 24 Hours)

| | | Gene Expression Level in | |
|---|---|---|---|
| | | Group with Water-soluble | |
| | | Nuclear Protein (Salmon Milt | |
| | | Extract) Added [Ratio] | |
| Gene | | Versus Placebo | P- |
| Name | Protein Functionality | Group | value |

Proteasome-related Gene Expression

| | | | |
|---|---|---|---|
| UBA1 | Ubiquitin Complex Enzyme | 2.07 | 0.0091 |
| | E2D1 | | |
| UBC5 | Proteasome Subunit β3 | 1.45 | 0.0472 |
| PRE3 | 20S Proteasome β1 Subunit; | 1.44 | 0.0045 |
| | Involved in Cleavage of C- | | |
| | terminal Side of Peptide Acidic | | |
| | Residues | | |
| PRE4 | 20S Proteasome β7 Subunit | 1.74 | 0.0001 |
| PRE6 | 20S Proteasome α4 Subunit | 1.78 | 0.0001 |
| PRE8 | 20S Proteasome α2 Subunit | 1.85 | 0.0002 |
| PUP1 | 20S Proteasome β2 Subunit | 1.55 | 0.0004 |

Autophagy-related Gene Expression

| | | | |
|---|---|---|---|
| ATG7 | E1-like Activating Enzyme | 1.84 | 0.0122 |

As shown in Table 3, it was confirmed that the group after a lapse of 24 hours after the addition of the water-soluble nuclear protein (salmon milt extract) to the yeast significantly increased the related gene expression of various degradation systems compared to the placebo group (not added).

As described above, the water-soluble nuclear protein (salmon milt extract) contributed to reducing the oxidative stress marker and increased the expression level of the genes related to the degradation systems of cells and the like, such as proteasome, autophagy, and apoptosis, in both humans and yeast. The results newly point to a possibility that a nuclear protein can contribute to accelerating degradation and the like of defective or damaged proteins, cell organelles such as mitochondria, and cells themselves and can be involved in recycling in which these degraded cells and the like are recycled to produce new cells and the like and a possibility that the nuclear protein can therefore further contribute to a reduction and suppression of oxidative stress and improvement of health.

1. A degradation system accelerator for recycling enhancement, containing
a water-soluble nuclear protein with a molecular weight of 250 to 15,000 obtained by treating fish milt with an enzyme.

2. (canceled)

3. The degradation system accelerator according to claim 1, wherein
the degradation system is a proteasome degradation system.

4. The degradation system accelerator according to claim 1, wherein
the degradation system is an autophagy degradation system.

5. The degradation system accelerator according to claim 1, wherein
the degradation system is apoptosis.

6. A health food for a degradation system accelerator for recycling enhancement, the health food containing the degradation system accelerator according to claim 1.

7. The health food according to claim 6, wherein
the health food has a form of a tablet, drinkable preparation, capsule preparation, granule preparation, powdered preparation, pill, or jelly preparation.

8. (canceled)

9. (canceled)

10. (canceled)

11. (canceled)

12. A response increasing agent for a proteasome-related gene containing a water-soluble nuclear protein with a molecular weight of 250 to 15,000 obtained by treating fish milt with an enzyme.

13. A response increasing agent for an autophagy-related gene containing a water-soluble nuclear protein with a molecular weight of 250 to 15,000 obtained by treating fish milt with an enzyme.

14. A response increasing agent for an apoptosis-related gene containing a water-soluble nuclear protein with a molecular weight of 250 to 15,000 obtained by treating fish milt with an enzyme.

15. A recycling enhancer for a cell, a cell organelle, or a protein, the recycling enhancer containing a water-soluble nuclear protein with a molecular weight of 250 to 15,000 obtained by treating fish milt with an enzyme.

16. A health food for a proteasome degradation system accelerator for recycling enhancement, the health food containing the degradation system accelerator according to claim 3.

17. A health food for an autophagy degradation system accelerator for recycling enhancement, the health food containing the degradation system accelerator according to claim 4.

18. A health food for an apoptosis degradation system accelerator for recycling enhancement, the health food containing the degradation system accelerator according to claim 5.

19. (canceled)

20. The health food according to claim 16, wherein
the health food has a form of a tablet, drinkable preparation, capsule preparation, granule preparation, powdered preparation, pill, or jelly preparation.

21. The health food according to claim 17, wherein
the health food has a form of a tablet, drinkable preparation, capsule preparation, granule preparation, powdered preparation, pill, or jelly preparation.

22. The health food according to claim 18, wherein
the health food has a form of a tablet, drinkable preparation, capsule preparation, granule preparation, powdered preparation, pill, or jelly preparation.
